(19) United States
(12) Patent Application Publication
(10) Pub. No.: US 2006/0118114 A1
(43) Pub. Date: Jun. 8, 2006

(54) SCENTED ANESTHESIA BREATHING CIRCUIT

(76) Inventor: Allen J. Hinkle, Lebanon, NH (US)

Correspondence Address: MICHAEL J. WEINS, 31 BANK STREET, LEBANON, NH, 03766, US

(21) Appl. No.: 10/904,930

(22) Filed: Dec. 6, 2004

(51) Int. Cl. A61M 16/00; A62B 7/00

(52) U.S. Cl. 128/204.22; 128/204.23; 128/204.18

(57) ABSTRACT

An anesthesia breathing circuit has an inspiratory tube, and expiratory tube, and a mask which communicates with both the inspiratory tube and the expiratory tube. The present invention resides in using a material which incorporates a scent-releasing agent to fabricate at least one element of the breathing circuit through which gas is fed to the mask from an anesthesia machine. In one preferred embodiment, the scented element is a segment that may be coupled to conventional elements of an anesthesia breathing circuit so as to form a part of the inspiratory tube; the scented element can then be uncoupled when the scent is no longer desired.

### BACKGROUND OF THE INVENTION

Classically, an anesthesia breathing circuit is employed for delivery of an anesthetic gas mixture from an anesthesia delivery machine to a patient, and for returning exhaled gases from the patient to the anesthesia delivery machine. An anesthesia breathing circuit typically has an inspiratory tube for delivery of gas from the anesthesia delivery machine to a patient, an expiratory tube for delivery of gases exhaled by the patient to the anesthesia delivery machine, a face mask for sealably covering the nose and mouth of the patient, and a means for communicating between the face mask and the inspiratory tube and the expiratory tube. The means for communicating frequently includes an elbow connecting between the face mask and the inspiratory and expiratory tubes. These elements have traditionally been formed as discrete elements, but could be formed either partially or entirely as an integral assembly to reduce or eliminate leakage between adjacent elements. The details of these circuits vary depending on the particular circuit selected. The circuits that are typically employed include circular circuits, where the inspiratory and expiratory tubes are separate and distinct, and Mapleson-type circuits, where the inspiratory tube terminates at or inside the expiratory tube. One commonly used variant of the Mapleson-type circuit is the Bain circuit, where a portion of the inspiratory tube resides within the expiratory tube. The Bain circuit has been further refined in the Universal F2 circuit offered by King Systems, where the inspiratory tube is completely housed within the expiratory tube and the partitioning of the inspiratory and expiratory gasses occurs in an anesthesia machine to which the expiratory and the inspiratory tube, which is housed therein, are connected.

A Bain type anesthesia circuit 10 is illustrated in FIG. 1. It has an inspiratory tube 12 which terminates within an expiratory tube 14 as it approaches a connector piece 16, which in turn attaches to an elbow 18 which feeds a face mask 20. The connector piece 16 and the elbow 18 provide means for communicating between the face mask 20 and the inspiratory and expiratory tubes (12, 14). This Bain type anaesthesia circuit 10 attaches to an anesthesia machine 22 that regulates the supply of gas to the inspiratory tube 12 and from the expiratory tube 14. Such anesthesia circuits have the advantage that they only require a single tube leading to the mask and are more convenient for use where additional tubing could further reduce the access to the area where surgery is to be performed, such as during surgery on the head or on pediatric patients. However, such circuits typically require greater skill to be used effectively, as they require the fresh gas flow to be carefully adjusted to ensure that adequate fresh gas reaches the patient.

A circular anesthesia circuit 50 is illustrated in FIG. 2. It has separate tubes forming an inspiratory tube 52 and an expiratory tube 54. These tubes (52 and 54) feed separately into a Y-shaped connector piece 56, which in turn feeds an elbow 58 and a mask 60. The circular anesthesia circuit 50 attaches to an anesthesia machine 62, which has connectors essentially similar to those of the anesthesia machine 22 shown in FIG. 1. The anesthesia machine 62 employed with the circular anesthesia circuit 50 has valves to control the flow of gas through the inspiratory tube 52 and the expiratory tube 54, and thus the circular anesthesia circuit 50 does not require stringent control of the fresh gas flow rate to avoid rebreathing of exhaled gasses.

Both types of circuits are currently in use, and in both cases the mask employed is frequently scented to block the pungent scent of the anesthetic gas used on the patient. The benefit of using a scented mask is set forth in Published Application 2003/127,102, and in U.S. Pat. Nos. 5,109,839 and 4,896,666. The latter patent is for the use of a mask in combination with a pacifier. The use of a pacifier has been found especially beneficial for pediatric use. The pacifier makes the infant more at ease and reduces the pulse, which hastens the speed of anesthetic induction.

The scent in the mask is released into dead air space and must diffuse through this space to mingle with the anesthesia gas mixture being fed to the patient. To provide an effective concentration of scent, relatively high concentrations of the scented material need to be incorporated into the mask. This substantially increases the cost of the masks. Furthermore, since the masks are provided in different sizes and since various gasses require different scents, a large inventory of masks must be maintained. While the inventory problem is, in part, addressed by the '102 application, it has done so at the costs of complicating the preparation of the anesthesia procedure as well as adding additional components which must be secured to the mask and which could create a hazard if they were to detach during use.

Another problem with scented masks is that the scented agent continues to release the scent after the patient has been anesthetized, and the scent is no longer needed. The volatile agents typically used to release the scent may make the anesthetic gas less effective, thus requiring higher concentrations to be used, and may also interfere with some types of gas monitoring systems which are employed to adjust the composition of the anesthetic gas mix provided to the patient.

Thus, there is a need for an alternative system for blocking the pungent smell of anesthetic gases which is more cost effective and more convenient for use. There is also a need for a system which allows the scent to be readily reduced or eliminated when no longer providing a benefit to the patient.

### SUMMARY OF THE INVENTION

The present invention is for an anesthesia breathing circuit of the type having an inspiratory tube, and expiratory tube, a mask, and a means for communicating between the mask and the inspiratory tube and the expiratory tube, which frequently includes a connector and an elbow. The details of two examples of such anesthesia circuits currently in use are discussed in greater detail above. The improvement resides in using a material which incorporates a scent-releasing agent to fabricate at least one element of the breathing circuit through which gas is fed to the mask. This scented element can be either a segment or the entirety of the inspiratory tube and/or the means for communication between the mask and the inspiratory tube and expiratory tube. In one preferred embodiment, the scented element is a segment that may be coupled to conventional elements of an anesthesia breathing circuit so as to form a part of the inspiratory tube. This allows the scented element to readily be uncoupled, either by diverting the gas flow or by removing the scented element, possibly by substituting a similar non-scented element, once the patient has been anesthetized and the scent is no longer needed.

In a preferred embodiment, a segment of the inspiratory tube is treated to provide the scent, this segment being configured to be insertable between a conventional inspiratory tube and an anesthesia machine. Preferably, the insertable segment terminates at a first connector configured to couple with the output coupling of the anaesthesia delivery machine and a second connector which is configured to match the connector of the output coupling of the anaesthesia delivery machine, thereby allowing the conventional inspiratory tube to be connected to the second connector.

It is further preferred that, when an insertable scented segment is provided which is configured to connect to the inspiratory tube, that this segment be corrugated to increase the area per unit length of the inspiratory tube, and thereby reduce the concentration of scent-releasing agent needed. This benefit is also present if a section of the inspiratory tube is scented; in the case of circular circuits using the standard corrugated tubing which is designed for flexibility without kinking, a length of about 12 inches (30 cm) should be satisfactory.

When the scented element is a segment of the inspiratory tube that is formed of standard corrugated tubing, means for compressing the sidewalls of the corrugated surface into intimate contact can be provided to substantially suppress the scenting of the gas.

Treating such a segment of the inspiratory tube provides a benefit over the treatment of the other elements in that the surface of the inspiratory tube is not exposed to gases exhaled from the lungs of the patient. Thus, there will be no moisture or other by-products from the exhaled gas flowing over the surface of the inspiratory tube which might reduce the effectiveness of the scented surface in transferring scent to the gas inspired by the patient.

### BRIEF DESCRIPTION OF THE FIGURES

FIG. 1 is view of a prior art Bain type anesthesia breathing circuit.

FIG. 2 is a view of a prior art circular anesthesia breathing circuit.

FIG. 3 is a view of an improved Bain type anesthesia breathing circuit showing the elements of the circuit illustrated in FIG. 1 that are subject to treatment with a scent-releasing agent to provide the improvement of the present invention.

FIG. 4 is a view of an improved circular anesthesia breathing circuit showing the elements of the circuit illustrated in FIG. 2 that are subject to treatment to provide the improvement of the present invention.

FIG. 5 is a section view of a section of an inspiratory tube of the improved circuit shown in FIG. 4, having a section length L. This tube is corrugated to increase flexibility without promoting kinking of the tube that could interrupt flow.

FIGS. 6 and 7 illustrate an assembly to be used in combination with a scented length L of the inspiratory tube to provide means for controlling scent release. FIG. 6 illustrates the scented length L_{E} in its elongated state, where it releases scent to the anaesthesia gas passing therethrough, while FIG. 7 illustrates the scented length L collapsed to L_{C} so as to minimize scent release.

FIGS. 8 through 10 illustrate an insertable segment of scented tubing having interfaces configured to slidably engage the connectors of the inspiratory port of the anesthesia machine and the standard coupling of the expiratory tube.

FIGS. 11 and 12 illustrate an insertable segment of scented tubing similar to that shown in FIGS. 8-10, which can be employed as a removable portion of the inspiratory tube to provide scent for either a Mapleson or Bain type breathing circuit, as shown in FIG. 11, or for a circular breathing circuit, as shown in FIG. 12.

FIG. 13 illustrates a branch segment of scented tubing similar to that shown in FIGS. 11-12, which can be employed as a removable portion of the inspiratory tube to selectively provide scent for either a circular breathing circuit, as shown, or a Mapleson or Bain type breathing circuit. The branch segment includes a valve that allows the scent to be eliminated without requiring disconnection of the elements of the breathing circuit.

### DETAILED DESCRIPTION OF THE PREFERRED EMBODIMENTS

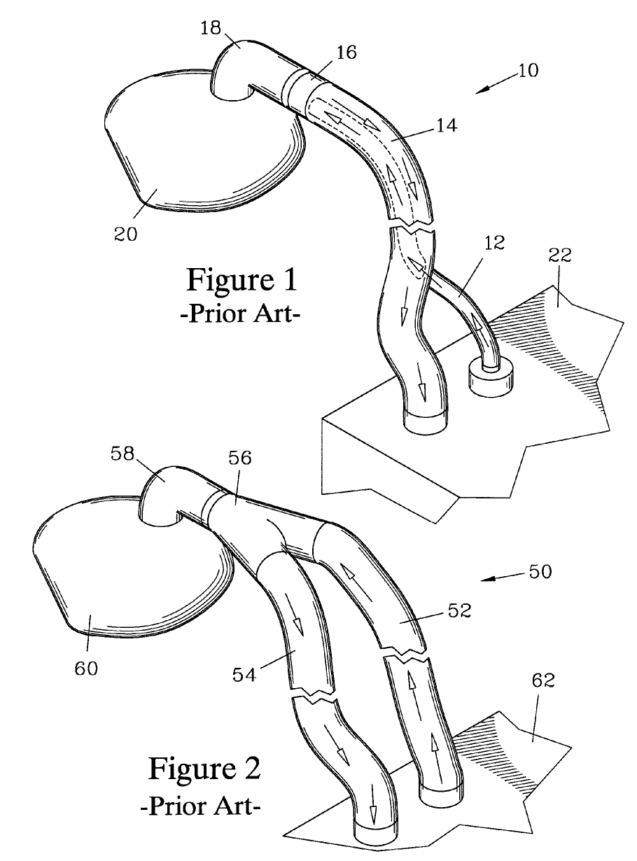

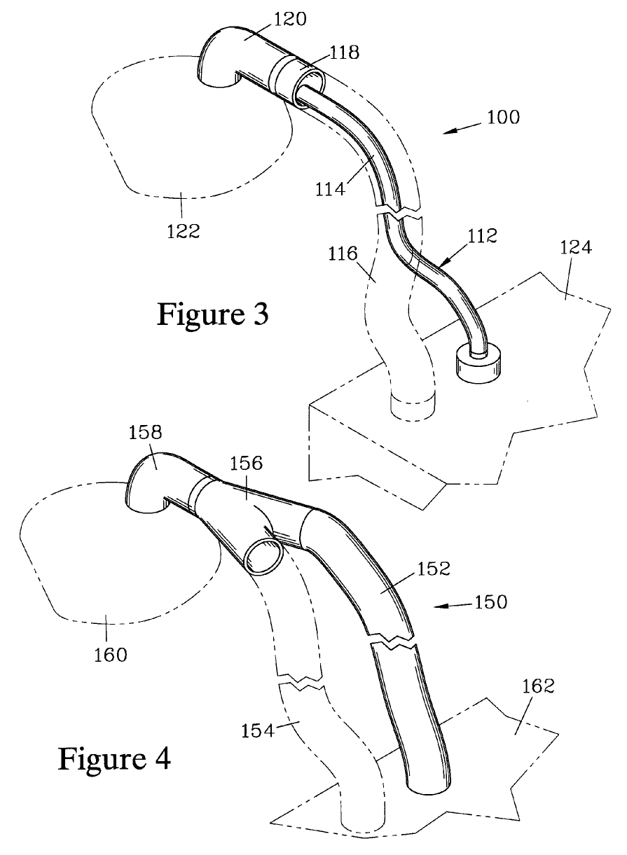

FIG. 3 is an illustration of a Bain type anesthesia circuit 100 of the present invention which has the same structural elements as the Bain type anesthesia circuit 10 illustrated in FIG. 1. It has an inspiratory tube 112 having a residential segment 114 that resides within an expiratory tube 116 and terminates in a terminal region 118 of the expiratory tube 116 which, in this circuit, serves as the connector piece that attaches the expiratory tube 116 to an elbow 120 which in turn connects to a mask 122, allowing anesthesia gas from the residential segment 114 of the inspiratory tube 112 to flow into the mask 122 and expired gas from a patient (not shown) to flow from the mask 122 into the expiratory tube 116. The terminal region 118 of the expiratory tube 116 and the elbow 120 of this embodiment serve as means for communicating between the mask 122 and the inspiratory tube 112 and the expiratory tube 116. Thus, in breathing circuits where a terminating segment of the expiratory tube extends beyond the end of the inspiratory tube, this terminating segment can also be considered as part of the means for communicating between the mask and the inspiratory and expiratory tubes.

The Bain type anaesthesia circuit 100 illustrated attaches to an anesthesia machine 124 that regulates the supply of gas to the inspiratory tube 112 and processes the gas from the expiratory tube 116. The improvement of the present invention is achieved by providing scent to one or more of the inspiratory tube 112, the terminal region 118, and the elbow 120 by fabricating at least a portion of the element from a material which incorporates a scent-releasing agent. Similar elements could be scented in other Mapleson type anesthesia circuits. The remaining elements of this embodiment (the expiratory tube 116 and the mask 122) are shown in phantom, since these elements can be the same as the prior art elements.

In this embodiment, the inspiratory tube 112 passes through the expiratory tube 116 and is sheathed by it in the region near the mask 122 where kinking is most likely to occur, which limits the problem of having the inspiratory tube 112 kink so as to cut off flow. Thus, corrugation of the tubing used for the inspiratory tube 112 is not a necessity. However, corrugation of the inspiratory tube 112 will benefit the circuit, since it will increase the surface area per unit length and thus reduce the concentration of scent-releasing agent needed in the tubing to effectively block the pungent odor of the anesthesia gas.

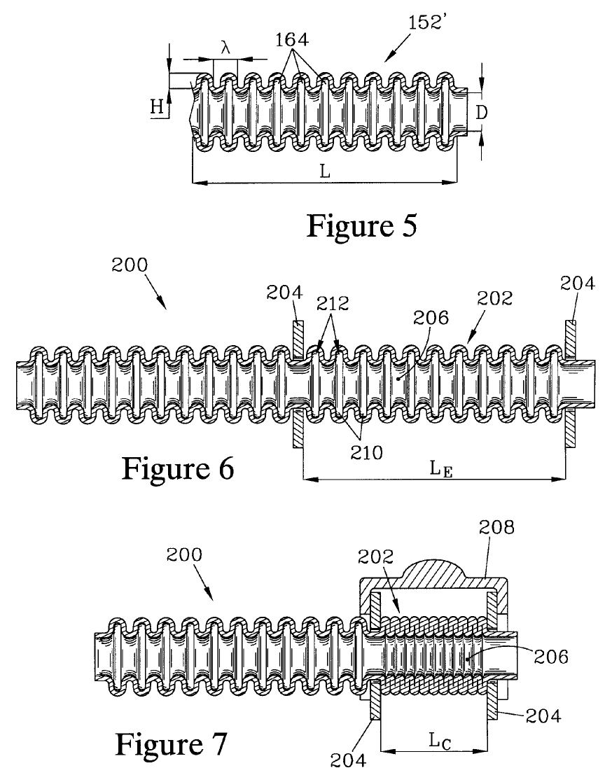

FIG. 4 is an illustration of a circular anesthesia circuit 150 which is similar to the circular anesthesia circuit 50 illustrated in FIG. 2. The circular circuit 150 has separate tubes forming an inspiratory tube 152 and an expiratory tube 154. These tubes (152 and 154) feed separately into a Y-shaped connector piece 156, which in turn feeds an elbow 158 and a mask 160. The Y-shaped connector piece 156 and the elbow 158 of this embodiment serve as means for communicating between the mask 160 and the inspiratory tube 152 and the expiratory tube 154. The circular anesthesia circuit 150 attaches to an anesthesia machine 162. The anesthesia machine 162 has internal valves (not shown) which regulate the flow through the inspiratory tube 152 and the expiratory tube 154 such that the inspiratory tube 152 will be fed anesthesia gas and, at the same time, a valve controlling flow in the expiratory tube 154 will be closed, thereby promoting flow of anaesthesia gas to the patient. Conversely, when the patient exhales, the valve controlling the flow in the inspiratory tube 152 is closed, blocking exhaled gas from traveling into inspiratory tube 152 while the valve in the expiratory tube 154 is open to allow expired gas to pass therethrough and into the asthenia machine 162. In this embodiment, it is preferred for the inspiratory tube 152 and the expiratory tube 154 to both be fabricated from corrugated tubing which increases flexibility while inhibiting kinking, as shown in FIG. 5 by the inspiratory tube 152′. Again, in the circular anesthesia circuit 150, the improvement of the present invention is achieved by providing scent to one or more of the inspiratory tube 152, the connector piece 156 and the elbow 158. The remaining elements of this embodiment are shown in phantom, since these elements can be the same as the prior art elements.

FIG. 5 shows a scented tubing segment of length L of the inspiratory tube 152′, illustrating details of the preferred corrugated structure, which is typically used to administer anesthesia. The length L illustrated in FIG. 5 is the neutral length of the segment, wherein the inspiratory tube 152′ is neither under a compressive load or a tensile load. The inspiratory tube 152′ typically has grooves 164 configured such that the ratio λ to H of the grooves 164 is in the range of about 1:1. This corrugated structure provides multiple benefits, one of which is to prevent kinking so that the flow will not be interrupted, as discussed above. The corrugations also increase the surface area of the inspiratory tube 152′ per unit length, and thus provide more surface contact with the anesthesia gas as it passes therethrough. In order for the corrugation to be effective, the grooves 164 of the corrugations should be relatively shallow so as not to create dead air spaces that will require the scent to diffuse therethrough. It is felt that the ratio of λ to H of the grooves 164 which is typically used in such tubing to avoid kinking should also serve to avoid dead spaces.

In the embodiments illustrated in FIGS. 3 and 4, there are multiple locations where the scent can be provided. However, if one were to scent only one of the elements, which could reduce costs and simplify manufacturing, a preferred element to form from a scented material would be the inspiratory tube or a segment thereof. The large surface area of this element, relative to the other elements that could be selected, should be likely to allow a lower concentration of scent-releasing agent to be used while still providing satisfactory performance.

A secondary advantage may arise from treating only the inspiratory tube or a section thereof. The scent so placed assures that, when gas flows over the scent-releasing surface, this flow is directed toward the mask, and thus all the entrained scent is advanced to the mask. When the patient exhales, the expelled gases from the lungs of the patient pass through the expiratory tube and the intermediate segments (the elbow and the connector piece), but do not flow into the inspiratory tube. In a Mapleson or Bain type circuit, a positive pressure is maintained in the inspiratory tube with respect to the expiratory tube, this pressure being sufficient to prevent back flow in the inspiratory tube as the patient exhales; in a circular circuit, valves on the anesthesia machine block back flow in the inspiratory tube as discussed in greater detail above. As a result, there is a potential advantage obtained by treating the inspiratory tube over treating the connecting piece or the elbow in that these intermediate segments see flow of both fresh gases and exhaled gases, the exhaled gases containing water vapor and other by-products generated by the respiratory process. These by-products may reduce the effectiveness of the scented surface in transferring scent to the gas being subsequently passed therethrough to the patient, by adversely affecting the activity of the surface and thereby reducing the release of scent. Also, the bidirectional use of the surface may deplete the mean concentration of scent molecules encountered by the anesthesia gas.

The ability to regulate the release of scent as a function of time is a great assistance in administering anesthesia to a patient. The use of scent is beneficial in reducing the distress of the patient when the gas is being administrated; however, the scent may also make the anesthesia less effective, which could require the use of higher concentration of the anesthesia gas. For this reason, it is preferred for the scent to be eliminated or greatly reduced once the patient has lost consciousness, thereby reducing the concentration of anesthesia needed. Additionally, the scent-releasing agent may interfere with accurate monitoring of the composition of the expired gases. FIGS. 6 and 7 illustrate an assembly to be used in combination with a scented segment of length L of the inspiratory tube to provide one means for controlling the scent release.

FIGS. 6 and 7 illustrate an inspiratory tube 200 which has a scented segment 202. The scented segment 202 is maintained between a pair of washers 204 that reside in a pair of spaced apart valleys that delineate the scented section 202 of the inspiratory tube 200, the washers serving to provide protrusions. In FIG. 6, the scented segment 202 is in its unconstrained condition and has an elongated length L_{E} where it has a ribbed inner surface 206 over which the anesthesia gas flows. The elongated length L_{E} will frequently be the same as the neutral length L; however, the elongated length L_{E} may be sightly different if the inspiratory tube 200 is in use and is either slightly compressed or in tension. The scented section 202 is treated with the scent-releasing agent and, when in its unconstrained state (where it is at its elongated length L_{E}), the ribbed inner surface 206 transfers scent to the gas passing thereby. FIG. 7 illustrates the same tube 200; however, the scented segment 202 has been re-configured to reduce or substantially eliminate the transfer of scent to the anesthesia gas. When the scent is to be reduced and, preferably, essentially eliminated, the elongated length L_{E} is collapsed to a collapsed length L_{C}, thereby reducing the scented region available for transfer of scent to the gas flowing through the inspiratory tube 200. The scented segment 202 is held in such position by a clip 208 that engages the washers 204. If the scent is to be substantially eliminated, it is preferred that only lower portions 210 (shown in FIG. 6) of grooves 212 that form the ribbed inner surface 206 be treated with the scent-releasing agent. If this is done, then collapsing the scented segment 202 essentially eliminates all the exposed scent-releasing regions. It should be noted that the scented segment 202 need not be the same diameter as the remainder of the inspiratory tube 200, and where it is desirable to reduce the length of the scented segment 202, such might be achieved by increasing the diameter to provide a greater surface area per unit length.

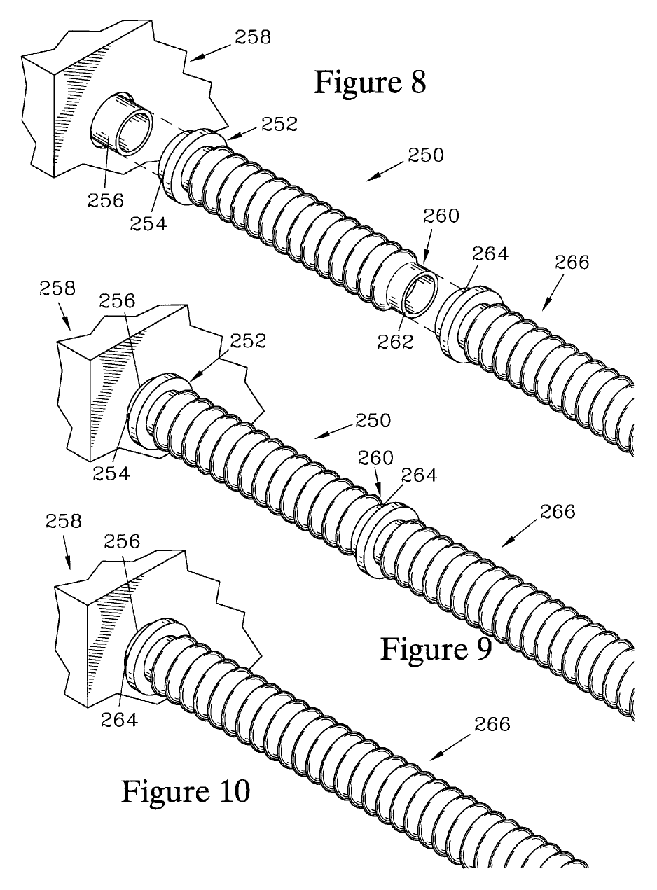

FIGS. 8 and 9 illustrate an insertable tubing segment 250 terminating in a first end 252 which has a first end coupling 254 designed to sealably engage a standard anesthesia inspiratory port 256 of an anesthesia machine 258. In this embodiment, the first end coupling 254 and the standard anesthesia inspiratory port 256 are configured so that they can be forced together with a sliding action such that their overlapping surfaces will form a seal. The scented tube 250 also terminates in a second end 260 which has a second end coupling 262 which is designed to sealably engage a standard inspiratory tube anaesthesia machine coupling 264 of an inspiratory tube 266. When interposed between the inspiratory tube 266 and the anesthesia machine 258 as shown in FIG. 9, the insertable tubing segment 250 essentially becomes a part of the inspiratory tube 266, forming a removable portion of the inspiratory tube, while the conventional inspiratory tube 266 forms a permanent portion.

The insertable tubing segment 250 allows the intermittent administration of scented anesthesia gas. The insertable tubing segment 250 is inserted when the anesthesia gas is first introduced and, after the patient has lost consciousness, the insertable tubing segment 250 can be removed from the circuit, and the standard inspiratory tube anaesthesia machine coupling 264 of the inspiratory tube 266 is connected directly to the standard anesthesia inspiratory port 256 of the anesthesia machine 258, as shown in FIG. 10. Removing the insertable tubing segment 250 when it is no longer needed eliminates the problem of continuous scenting of the anesthesia gas. The length and diameter of the insertable tubing segment 250 should be chosen to provide sufficient surface area for adequate transfer of scent while retaining the desired degree of flexibility of the insertable tubing segment 250. If standard tubing such as is currently employed for circular anesthesia breathing circuit tubes is used, then a length L of about 12 inches (30 cm) should be adequate. The insertable tubing segment 250 could alternatively be placed between the inspiratory tube 266 and a connector piece, but such placement may make removal of the insertable tubing segment 250 more difficult, since it would require access space close to the patient.

It should be noted that the scent could alternatively be reduced by collapsing the insertable tubing segment 250 and retaining it in such collapsed state, in the manner discussed above with regard to the scented segment 200 shown in FIGS. 6 and 7. This would allow reducing or substantially eliminating the scent without requiring the disconnection and reconnection of elements of the breathing circuit.

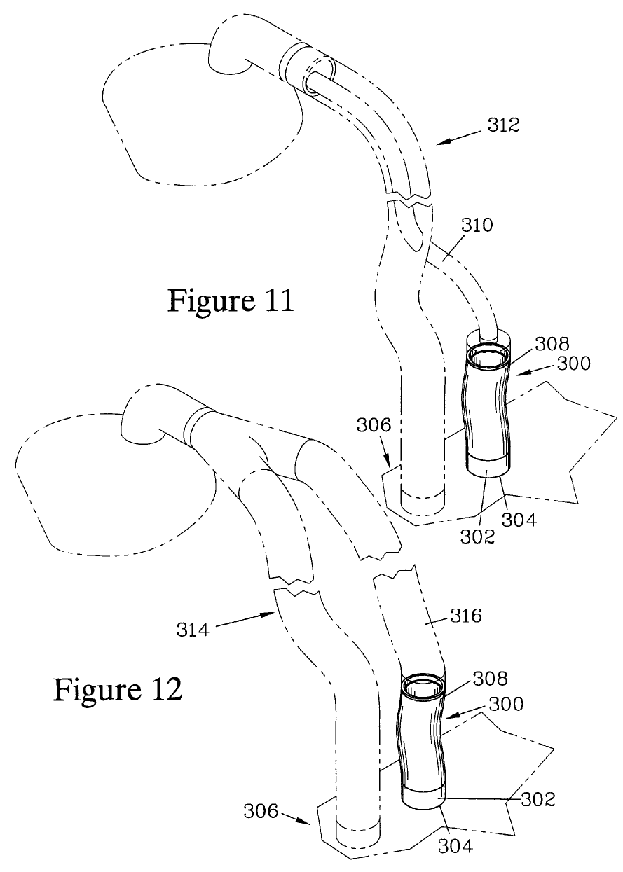

FIGS. 11 and 12 illustrate an insertable tubing segment 300 which is functionally similar to the insertable tubing segment 250 shown in FIGS. 8-10. The insertable tubing segment 300 again has a first end coupling 302 configured to be connected to a standard anesthesia inspiratory port 304 of an anesthesia machine 306, and a second end coupling 308 which has a configuration identical to that of the standard anesthesia inspiratory port 304. An inspiratory tube 310 of a Mapleson type anesthesia circuit 312 (a Bain circuit is illustrated in FIG. 11) can be connected to the second end coupling 308, when scent is desired, or directly to the standard anesthesia inspiratory port 304, when no scent is desired. Similarly, when a circular anesthesia circuit 314 is to be employed, an inspiratory tube 316 of the circular anesthesia circuit 314 can be connected to either the second end coupling 308, as shown in FIG. 12, or directly to the standard anesthesia inspiratory port 304. Thus, the insertable tubing segment 300 can be employed with either type of anesthesia circuit, simplifying the maintenance of inventory. As an alternative to removing the insertable tubing segment 300, it could be made collapsible to allow retaining the insertable tubing segment 300 in a collapsed state to reduce the release of scent.

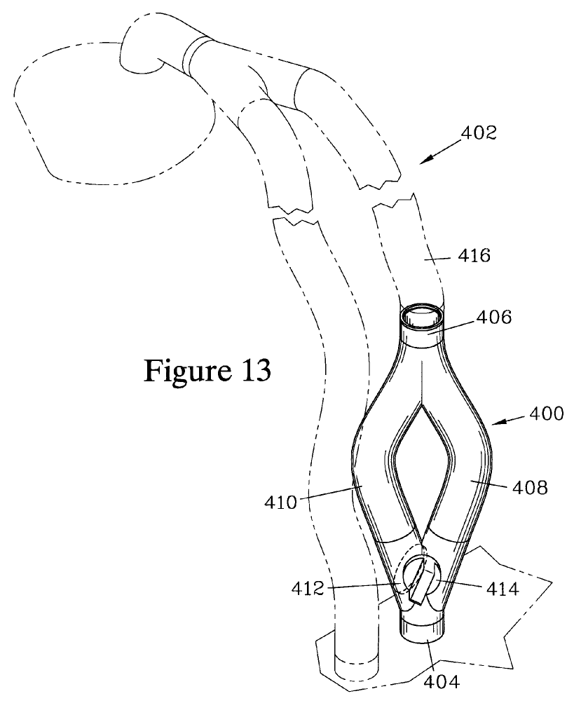

FIG. 13 illustrates a scented branched tubing segment 400 which can be employed in a manner similar to that of the insertable tubing segments 250 and 300 shown in FIGS. 8-12; however, the scented branched tubing segment 400 allows the scent to be removed without disconnection. FIG. 13 illustrates the scented branched tubing segment 400 employed with an anesthesia breathing circuit 402, shown in phantom. While a circular breathing circuit is shown, the scented branched tubing segment 400 could be used with a Mapleson or Bain type breathing circuit in a manner similar to that of the insertable tubing segment 300 as shown in FIG. 11.

The scented branched tubing segment 400 terminates in a first end coupling 404 and a second end coupling 406. The scented branched tubing segment 400 has a first branch 408, which is formed at least partially from a scented material, and a second branch 410, which is formed from a non-scented material. The scented branched tubing segment 400 also has a valve 412 located near the first end coupling 404. The valve 412 can be operated by use of a knob 414 to direct air flowing into the first end coupling 404 either through the first branch 408, as shown, or through the second branch 410. From either of the branches (408, 410), the air flows to the second end coupling 406, and thereafter into an inspiratory tube 416 of the anesthesia breathing circuit 402.

It should be appreciated that either scented connector pieces or elbows could be replaced with non-scented equivalents to create intermittent introduction of scent, subject to the limitation of the effectiveness of these scented elements discussed above and the need to allow access close to the patient to replace the scented element.

While the novel features of the present invention have been described in terms of particular embodiments and preferred applications, it should be appreciated by one skilled in the art that substitution of materials and modification of details obviously can be made without departing from the spirit of the invention.

1. An improved anesthesia breathing circuit for delivery of anesthetic gas from an anesthesia delivery machine to a patient, the anesthesia breathing circuit having:
an inspiratory tube for delivery of gas from the anesthesia delivery machine;
an expiratory tube for delivery of gases expired by the patient to the anesthesia delivery machine;
a face mask for sealably covering the nose and mouth of the patient, the face mask having a scenting level which is ineffective to block the pungent odor of the anaesthesia gas mix; and
means for communicating between the inspiratory tube and the expiratory tube, this means also communicating with and connecting to the face mask;
the improvement comprising;
fabricating at least one of the inspiratory tube and the means for communicating between the inspiratory tube and the expiratory tube and communicating with and connecting to the face mask, at least in part, from a scented material.

2. The improved anesthesia breathing circuit of claim 1 wherein the means for communicating between the inspiratory tube and the expiratory tube and communicating with and connecting to the face mask further comprises:
a connector piece fed separately by the expiratory tube and the inspiratory tube and communicating with the mask.

3. The improved anesthesia breathing circuit of claim 15 wherein the elbow is formed from a scented material.

4. The improved anesthesia breathing circuit of claim 15 wherein the inspiratory tube is corrugated and at least a portion of the corrugated inspiratory tube is fabricated from a scented material.

5. The improved anesthesia breathing circuit of claim 1 wherein the inspiratory tube is corrugated and at least a portion of the corrugated inspiratory tube is fabricated from a scented material.

6. (canceled)

7. The improved anesthesia breathing circuit of claim 2 wherein the mask is scentless and the inspiratory tube is formed with at least a segment formed from a corrugated tube which is scented over a portion having an in-service elongated length L_{E}, the anesthesia breathing circuit further comprising:
means for shortening the elongated length L_{E} of said scented portion to an extent that the corrugated tubing has grooves that are reduced to an effective groove length λ approaching zero,
thereby causing a reduction in the scent when said scented portion is so shortened.

8. The improved anesthesia breathing circuit of claim 7 wherein said means for shortening the elongated length L_{E} of said scented portion further comprises:
a pair of protrusions located at the ends of said scented portion; and
a clip which can be engaged with said pair of protrusions when said scented portion is collapsed to bring said pair of protrusions to a reduced separation, said clip being configured to maintain said pair of protrusions at said reduced separation when engaged therewith.

9. The improved anesthesia breathing circuit of claim 1 wherein at least a portion of the inspiratory tube forms an external inspiratory tube segment which is external to the expiratory tube and further wherein the external segment of the inspiratory tube includes a removable portion, which is fabricated from a scented material, and a permanent portion which communicates with the means for communicating between the inspiratory tube and the expiratory tube and communicating with and connecting to the face mask.

10. The improved anesthesia breathing circuit of claim 2 wherein the inspiratory tube includes a removable portion which is fabricated from a scented material, and a permanent portion which communicates with the means for communicating between the inspiratory tube and the expiratory tube and communicating with and connecting to the face mask.

11. The improved anesthesia breathing circuit of claim 9 wherein the permanent portion of the inspiratory tube terminates at a standard inspiratory tube anesthesia machine coupling which is configured to sealably engage a standard anesthesia inspiratory port of the anesthesia delivery machine,
further wherein said removable portion terminates in a first end coupling, which is configured to sealably engage the standard anesthesia inspiratory port of the anesthesia delivery machine, and a second end coupling, which is configured to sealably engage the standard inspiratory tube anesthesia machine coupling of the permanent portion.

12. The improved anesthesia breathing circuit of claim 10 wherein the permanent portion of the inspiratory tube terminates at a standard inspiratory tube anesthesia machine coupling which is configured to sealably engage a standard anesthesia inspiratory port of the anesthesia delivery machine,
further wherein said removable portion terminates in a first end coupling, which is configured to sealably engage the standard anesthesia inspiratory port of the anesthesia delivery machine, and a second end coupling, which is configured to sealably engage the standard inspiratory tube anesthesia machine coupling of the permanent portion.

13. The improved anesthesia breathing circuit of claim 11 wherein said removable portion further comprises;
a first branch formed, at least in part, from a scented material;
a second branch formed from an unscented material; and
a valve for selectively communicating either of said first branch or said second branch with said first end coupling and said second end coupling.

14. The improved anesthesia breathing circuit of claim 12 wherein said removable portion further comprises:
a first branch formed, at least in part, from a scented material;
a second branch formed from an unscented material; and
a valve for selectively communicating either of said first branch or said second branch with said first end coupling and said second end coupling.

15. The improved anesthesia breathing circuit of claim 2 wherein the means for communicating between the inspiratory tube and the expiratory tube and communicating with and connecting to the face mask further comprises:
an elbow connecting the connector piece to the mask.

16. The improved anesthesia breathing circuit of claim 1 wherein the means for communicating between the inspiratory tube and the expiratory tube and communicating with and connecting to the face mask further comprises:
a connector piece wherein the connector piece is connected to the expiratory tube and communicates with the inspiratory tube.

17. The improved anesthesia breathing circuit of claim 16 wherein the means for communicating between the inspiratory tube and the expiratory tube and communicating with and connecting to the face mask further comprises:
an elbow for connecting the mask to the connector piece.

18. The improved anesthesia breathing circuit of claim 2 wherein the mask is scentless, the improvement further comprising:
means for reducing the scenting of the anesthesia breathing circuit.

19. The improved anesthesia breathing circuit of claim 16 wherein the mask is scentless, the improvement further comprising:
means for reducing the scenting of the anesthesia breathing circuit.

20. An improved anesthesia breathing circuit for delivery of anesthetic gas from an anesthesia delivery machine to a face mask configured to be worn by a patient and for delivery of gases expired by the patient from the face mask to the anesthesia delivery machine, the anesthesia breathing circuit having:
an inspiratory tube for delivery of gas from the anesthesia delivery machine;
an expiratory tube for delivery of gases expired by the patient to the anesthesia delivery machine; and
means for communicating between and connecting to the face mask and the inspiratory tube and the expiratory tube;
the improvement comprising:
at least one of the inspiratory tube and the means for communicating and connecting to, at least in part, being scented.
